(19) United States
(12) Patent Application Publication
(10) Pub. No.: US 2014/0275948 A1
(43) Pub. Date: Sep. 18, 2014

(54) INFORMATION TERMINAL DEVICE

(71) Applicant: Funai Electric Co., Ltd., Osaka (JP)

(72) Inventor: Shinichi KAMISOYAMA, Daito-shi (JP)

(73) Assignee: Funai Electric Co., Ltd., Osaka (JP)

(21) Appl. No.: 14/202,410

(22) Filed: Mar. 10, 2014

(30) Foreign Application Priority Data
Mar. 18, 2013 (JP) 2013-054794

(51) Int. Cl. A61B 5/00

(52) U.S. Cl. CPC A61B 5/444
USPC 600/407

(57) ABSTRACT

An information terminal device includes an imaging unit which captures a face image of a user, a main memory which extracts and stores an eyeball area and an under eye region from the face image, and a control unit which detects an amount of color change in a pupil and iris color at a current point in time relative to the pupil and iris color in the eyeball area at a past point in time after factoring in an amount of color change from face skin color in the entire face image that includes the eyeball area and the undereye region captured by the imaging unit at the current point in time compared to the entire face image that includes the eyeball area and the under eye region captured at a past point in time.

### BACKGROUND OF THE INVENTION

1. Field of the Invention

The present invention relates to an information terminal device and particularly to an information terminal device including an imaging unit which captures images that include a target diagnostic region in a living body.

2. Description of the Related Art

Imaging devices equipped with imaging units that capture images that include target diagnostic regions in living bodies have been known in the past. For example, see Japanese Patent Application Laid-Open Publication No. 2004-329620.

Japanese Patent Application Laid-Open Publication No. 2004-329620 discloses an imaging device equipped with a CCD (imaging unit) that captures the user's facial image. This imaging device is configured such that diagnostic data for self-diagnosis of changes (degree of improvement) in the user's physical condition, symptoms and the like is generated by comparing images of specific regions to each other such as the white of the eye or undereye area of the same user captured on different dates and times. Note that the generation of the diagnostic data involves the use of the results of comparison between the hue of a specific region such as the white of the eye or undereye area of a past image and the hue of a specific region such as the white of the eye or undereye area of the current image. Specifically, diagnostic data is generated based on the amount of color change from past to current in the yellow component, for example, that is present in a specific region within the images. Then, the constitution is such that diagnostic data is displayed on a display unit.

However, with the imaging device recited in Japanese Patent Application Laid-Open Publication No. 2004-329620, the target of hue comparison is limited only to specific regions (white of the eye, undereye area, etc.) within the image captured by the CCD, so there is a possibility that the amount of change in the hue of the specific regions includes not only factors of change directly related to the user's physical condition, symptoms, or the like (degree of improvement), but also factors not related to physical condition or symptoms, such as skin tanning effects or whitening effects due to cosmetics (skin care). However, this point is not taken into consideration in Japanese Patent Application Laid-Open Publication No. 2004-329620, so there is the problem of not being able to perform accurate self-diagnosis involving health management.

### SUMMARY OF THE INVENTION

Accordingly, preferred embodiments of the present invention provide an information terminal device which allows the user to accurately perform self-diagnosis involving health management.

An information terminal device according to a preferred embodiment of the present invention includes an imaging unit which captures images that include a target diagnostic region in a living body; a diagnostic data extraction unit which extracts diagnostic data for the target diagnostic region from the images captured by the imaging unit; a storage unit which stores the images captured by the imaging unit and the diagnostic data extracted by the diagnostic data extraction unit; and a detecting unit which detects the amount of color change in the target diagnostic region from first diagnostic data and second diagnostic data newer than the first diagnostic data that are stored in the storage unit after accounting for color changes in the entirety of an image that includes the target diagnostic region captured by the imaging unit at the current point in time compared to the entirety of an image that includes the target diagnostic region captured at a past point in time.

As was described above, as a result of the information terminal device according to a preferred embodiment of the present invention being equipped with a detecting unit which detects the amount of color change in the target diagnostic region from first diagnostic data and second diagnostic data newer than the first diagnostic data that are stored in the storage unit after accounting for color changes in the entirety of an image that includes the target diagnostic region captured by the imaging unit at the current point in time compared to the entirety of an image that includes the target diagnostic region captured at a past point in time, the amount of color change in the target diagnostic region constitutes an amount of color change that has factored in changes in the colors of the entirety of the image that includes the target diagnostic region from past to current. Therefore, the amount of change in color of the target diagnostic region is detected based on, in addition to color-changing factors directly related to the user's physical condition, symptoms, and the like, color changes derived from other factors as well such as skin tanning and whitening from cosmetics (skin care). Accordingly, accuracy and precision are increased when generating information pertaining to health management and the like based on the amount of color change in the target diagnostic region, so users can use this information terminal device to accurately perform self-diagnosis involving health management.

It is preferable that the information terminal device according to a preferred embodiment of the present invention also includes a generating unit which generates health management information for the living body based on the amount of color change detected by the detecting unit. Thus, the user can easily perform self-diagnosis involving health management based on health management information generated by the generating unit.

In such cases, it is preferable that the device also includes a determining unit which determines health status based on the health management information. If such a constitution is adopted, the user can obtain health status diagnostic results that are more accurate and precise with the use of this information terminal device because not only self-diagnosis by the user, but a health status evaluation performed by the determining unit based on the health management information is also added.

In the constitution further including the generating unit which generates health management information, it is preferable that the generating unit be configured to generate health management information of the living body based on the results of detection by the detecting unit of amounts of change in color in the target diagnostic region in the second diagnostic data relative to the first diagnostic data after factoring in color changes in the skin of the living body in the entirety of the image that includes the target diagnostic region captured at the current point in time compared to the entirety of an image that includes the target diagnostic region captured at a past point in time. By adopting such a constitution, health management information is generated based on the amount of color change in the target diagnostic region detected after factoring in change in the skin color of the living body from past to current images that include the target diagnostic region in their entirety. In other words, there is a possibility that various factors involved in changing the skin color of living bodies play a significant role in changing the color of the target diagnostic region, so by factoring in change in skin color, more accurate and precise health management information is generated.

In the constitution in which the detecting unit detects color change amounts in the target diagnostic region after factoring in skin color changes, it is preferable that the detecting unit be configured to detect amounts of color change in the target diagnostic region in the second diagnostic data relative to the first diagnostic data by comparing an image that includes the target diagnostic region captured at a past point in time with an image that includes the target diagnostic region captured at the current point in time and corrected to exclude the effects of color change in the target diagnostic region arising from changes in skin color from the past point in time to the current point in time. If such a constitution is adopted, out of factors that can cause changes in skin color, various factors such as skin tanning effects or whitening effects due to cosmetics (skin care) are eliminated in advance over the entirety of the images that include the target diagnostic region whose past and current images are to be compared, so it is possible to accurately ascertain the amount of color change (net color change amount) in the target diagnostic region at the current point in time relative to a past point in time under conditions that exclude factors affecting skin color change such as tanning or whitening not directly involved in the user's health management. As a result, health management information that enables accurate self-diagnosis is easily generated.

In the constitution in which the detecting unit detects color change amounts in the target diagnostic region after factoring in skin color changes, it is preferable that the device be configured to calculate the amount of color change of the skin of the living body in the entirety of the image that includes the target diagnostic region based on the entirety of the image as an achromatic image that includes the target diagnostic region at a past point in time and the entirety of the image as an achromatic image that includes the target diagnostic region at the current point in time. With such a constitution, the amount of color change in the skin of the living body is easily calculated based on the brightness (darkness) of the entirety of images including achromatic color including white, black, and their intermediate colors (grays), from which color components have been removed. Furthermore, because image processing such as that described above involves handling of achromatic image data, the processing load on the information terminal device is significantly reduced compared to the case of handling color image data.

In the constitution also including the generating unit which generates health management information, it is preferable that the generating unit be configured to generate the health management information according to amounts of change in the color of the target diagnostic region when the amount of color change in the target diagnostic region in the second diagnostic data relative to the first diagnostic data exceeds a specified threshold value. If such a constitution is adopted, health management information can be generated only when the amount of color change in the target diagnostic region exceeds a threshold, and no health management information is generated when the amount of color change in the target diagnostic region does not meet the threshold. That is, without being excessively sensitive to color change amounts in the target diagnostic region that can normally be ignored (generating erroneous health management information), it is possible to generate only health management information that is genuinely necessary for color change amounts of the target diagnostic region and that cannot be ignored, so more accurate and precise health management information is provided to the user.

In the constitution including the generating unit which generates health management information, it is preferable that the detecting unit be configured to detect amounts of change in color in the target diagnostic region of the second diagnostic data relative to the first diagnostic data by comparing the individual color scale values corresponding to the three primary colors of light at the target diagnostic region captured at a past point in time and the individual color scale values corresponding to the three primary colors of light at the target diagnostic region captured at the current point in time, for each of the colors. As a result, the amount of color change of the image of the target diagnostic region preferably is detected (ascertained) using three amounts of change as indexes, i.e., amount of red change, amount of green change, and amount of blue change, corresponding to the three primary colors of light in the target diagnostic region between past and present. That is, such color change amounts are easily ascertained in the image processing performed by the information terminal device.

In this case, it is preferable that the constitution be such that the amounts of change in color in the target diagnostic region of the second diagnostic data relative to the first diagnostic data are detected by the detecting unit by comparing the respective average values for the individual color scale values corresponding to the three primary colors of light at the target diagnostic region captured at a past point in time and the respective average values for the individual color scale values corresponding to the three primary colors of light at the target diagnostic region captured at the current point in time, for each of the colors. By adopting such a constitution, the amount of data used in comparison between past and current images is decreased by using the respective average values for the individual color scale values of the individual pixels compared to the case of detecting (ascertaining) the amount of change in the individual color scale values (red, green, and blue) in units of the individual pixels that make up the images in which the target diagnostic region (area) is captured. This makes it possible to significantly reduce processing load on the information terminal device and to perform processing rapidly.

In the constitution including the generating unit which generates health management information, it is preferable that the detecting unit be configured to be able to detect pigmented spots or tumorous areas which are present in the living body at the current point in time but were not present at a past point in time in the image that includes the target diagnostic region, and that the generating unit be configured to generate health management information for the living body by accounting for information on the pigmented spots or tumorous areas detected by the detecting unit. If such a constitution is adopted, not only is the amount of simple change in color in the target diagnostic region (area) made available as a basis of decision to generate health management information, but health management information is also generated after simultaneous detection (identification) of newly present pigmented spots or tumorous areas in the living body from the amount of color change. Consequently, the user can be provided with more realistic (practical) health management information germane to the user's health management.

In such cases, it is preferable that the image that includes the target diagnostic region captured by the imaging unit be a color image, and that the detecting unit be configured to detect the appearance of the pigmented spots and tumorous areas in the living body based on a composite image which superimposes a first inverted image that inverts the white and black portions of the entirety of an image that includes the target diagnostic region at a past point in time converted from the color image to an achromatic image and a second inverted image that inverts the white and black portions of the entirety of an image that includes the target diagnostic region at the current point in time. With such a constitution, pigmented spots or tumorous areas newly present on the living body are easily and precisely identified in image processing which uses a composite image that superimposes the first inverted image and the second inverted image.

In an information terminal device according to a preferred embodiment of the present invention, it is preferable that the imaging unit be configured such that the type of environmental light when capturing images that include the target diagnostic region can be input, and that the detecting unit be configured to detect the amount of color change of the target diagnostic region in the second diagnostic data relative to the first diagnostic data by comparing the image that includes the target diagnostic region captured at a past point in time and the image that includes the target diagnostic region captured at the current point in time after performing color correction on the image that includes the target diagnostic region captured at a past point in time and/or the image that includes the target diagnostic region captured at the current point in time based on the type of environmental light that is input at each point in time. By adopting such a constitution, the conditions involving environmental light at the time of imaging (imaging conditions) at individual points in time can be matched to the same status in the entirety of the images that include the target diagnostic region for which past and current images are compared to each other. This makes it possible to accurately ascertain the amount of color change in the target diagnostic region at the current point in time compared to the target diagnostic region at a past point in time.

As was described above, with various preferred embodiments of the present invention, the user is able to accurately perform self-diagnosis involving health management.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a perspective view showing a manner in which a user uses the information terminal device according to a preferred embodiment of the present invention to image a photograph of his own face for the purpose of health management.

FIG. 2 is a plan view showing the constitution of the information terminal device according to a preferred embodiment of the present invention.

FIG. 3 is a block diagram showing the constitution to control the information terminal device according to a preferred embodiment of the present invention.

FIG. 4 is a diagram showing a state in which a guide screen is displayed on the display unit in imaging mode in the information terminal device according to a preferred embodiment of the present invention.

FIG. 5 is a model diagram that illustrates image data processing in which the change in color between a past face image and the current face image that are captured is ascertained in the information terminal device according to a preferred embodiment of the present invention.

FIG. 6 is a diagram showing one example of health management information generated in the information terminal device according to a preferred embodiment of the present invention.

FIG. 7 is a model diagram for illustrating image data processing in which pigmented spots or tumorous areas appearing on the skin are identified based on the change in color between a past face image and the current face image that are captured in the information terminal device according to a preferred embodiment of the present invention.

FIG. 8 is a diagram showing a settings screen that is used when the type of environmental light at the time of imaging is set in advance for the information terminal device according to a preferred embodiment of the present invention.

FIG. 9 is a diagram that illustrates the flow of control by the control unit when application software having health management functions is executed in the information terminal device according to a preferred embodiment of the present invention.

### DETAILED DESCRIPTION OF THE PREFERRED EMBODIMENTS

Preferred embodiments of the present invention will be described below based on the drawings.

First, the constitution of the information terminal device 100 according to a preferred embodiment of the present invention will be described with reference to FIGS. 1 through 8.

The information terminal device 100 according to a preferred embodiment of the present invention, as shown in FIG. 1, preferably is a tablet-style terminal device that has a specified thickness (thickness in the Z direction) and preferably has a shape of a thin plate or substantially the shape of a thin plate. Furthermore, the information terminal device 100 has a shape and weight that make it easily portable by a user 1, and it is configured such that it can be used either indoors or outdoors depending on the user's location. In such cases, it can be used either by being completely held by the user 1 or in a state in which the device main body is placed on a resting surface such as a desk (not shown). Note that the user 1 is one example of the “living body” according to a preferred embodiment of the present invention.

The information terminal device 100 includes a case 10 made of plastic or metal formed in a specified shape and a display unit 11 including an LCD (liquid crystal display) embedded in the inner side of a frame portion 10a on the front side (Z2 side) of the case 10. Moreover, a protective film (not shown) having transparency and made of plastic is attached to the outermost surface of the display unit 11.

In addition, the information terminal device 100, as shown in FIG. 2, is equipped with an electrostatic capacitive touch panel portion 11a installed on the front surface side (front side on the plane of page) of the display unit 11, an imaging unit 12 installed on a side (Y1 side) of the display unit 11 with built-in CCD sensor or CMOS sensor (imaging element), an illuminance sensor 13 that is installed in the vicinity (X1 side) of the imaging unit 12 and senses ambient environmental light, a communication unit (see FIG. 3) that includes a built-in antenna 14a (see FIG. 3) and sends and receives electromagnetic waves for communications, a control circuit unit 15 (see FIG. 3) that is built into the case 10 and controls the information terminal device 100, a power supply unit 16 (see FIG. 3) that supplies power to the control circuit unit 15, a speaker 17 that is installed on a side (X2 side) of the display unit 11 and outputs sounds, and a group of operating buttons 18 installed on a side (X1 side) of the display unit 11.

The operating button group 18 includes a single plus key 18a and a plurality of button keys 18b. In the operation of the information terminal device 100, as shown in FIG. 2, the user 1 moves a cursor displayed within the display unit 11 while pushing the plus key 18a to the top, bottom, left, or right to select a variety of button icons, windows, and the like, and then presses the plus key 18a in that position. The user's intentions are thus reflected in the operation of application software. Furthermore, it is configured such that application software shutdown (termination), switching operations, and the like can be performed by pressing individual keys among the plurality of button keys 18b.

Moreover, the control circuit unit 15, as shown in FIG. 3, is equipped with a control unit 15a that includes a CPU and that controls the information terminal device 100, flash memory (ROM) 15b that stores control programs and the like executed by the control unit 15a, main memory (RAM) 15c used as a working memory that temporarily holds control parameters and the like that are used when control programs are executed, and an imaging signal processing unit 15d that converts images of the photographed object captured by the imaging unit 12 into image signals.

Here, in the present preferred embodiment, the constitution is such that the face 2 of the user 1 can be captured using the imaging unit 12 by performing specified operations in a state in which the user 1 holds the information terminal device 100 with the display unit 11 facing the front side (Z2 side facing the user 1) as shown in FIG. 1. Then, the constitution is such that health management information for the user 1 is generated based on the results of detecting the color of the face image 30 that captures the face 2 (color change detection result) as a result of operational processing by the control unit 15a (see FIG. 3), and also such that its content is displayed on the display unit 11 in a form like that of message (see FIG. 6). In addition, as shown in FIG. 6, the message is the health management information and includes the determination result from determining the health status based on the amount of color change (operational processing result) in the face image 30 by the control unit 15a. Accordingly, the user 1 can continue managing their health themselves, with reference to the message 91 (health management information) displayed on the display unit 11. Note that the face 2 is one example of the “target diagnostic region” according to a preferred embodiment of the present invention. Furthermore, the face image 30 is one example of the “image that includes the target diagnostic region” according to a preferred embodiment of the present invention. Moreover, the control unit 15a is one example of the “detecting unit,” “generating unit,” and “determining unit” according to a preferred embodiment of the present invention, and the message 91 is one example of the “health management information” according to a preferred embodiment of the present invention.

In addition, the present preferred embodiment is configured such that it is possible to ascertain via the control unit 15a (see FIG. 3) the color (color change) of each specific area, such as an image 30a that captures the portion of the eyeball areas (right eyeball area and left eyeball area) 2a and an image 30b that captures the portion of the undereye regions (right undereye region and left undereye region) 2b which constitute the face 2 included in the face image 30. Furthermore, it is configured such that health management information for the user 1 is generated based on color information (color change information) individually ascertained for each specific area such as the image 30a (portion of the eyeball area 2a) and the image 30b (portion of the undereye region 2b). Here, the face image 30 (the images 30a and 30b) refers to images captured by the imaging unit 12 at any time (see FIG. 1). Accordingly, the face image 30 will be explained below separately for a face image 31 (images 31a and 31b) which is captured at a past point in time in relative terms and a face image 32 (images 32a and 32b) which is captured at the current point in time in relative terms. Note that the eyeball area 2a and the undereye region 2b that partially make up the face 2 constitute examples of the “target diagnostic region” according to a preferred embodiment of the present invention. Moreover, the images 30a and 30b constitute examples of the “image that includes the target diagnostic region” according to a preferred embodiment of the present invention.

The operation contents of the control unit 15a (contents of control pertaining to image data processing) will be described in detail below from imaging of the face 2 by the imaging unit 12 (capture of the face image 30) to generation of health management information and display of the message 91 or the like on the display unit 11.

A variety of application software is executed on the information terminal device 100. Specifically, the application software stored on the flash memory 15b (see FIG. 3) includes application software which images the face 2 of the user 1 and provides specified health management information to the user 1 based on changes in the color of the face image 30 captured as image data.

First, as shown in FIG. 2, the user 1 (see FIG. 1) starts application software that provides health management information (the health management application) by touching specified locations on the touch panel portion 11a or pressing specified button keys within the operating button group 18. Then, startup of this application software causes the control unit 15a to drive the imaging unit 12 in the information terminal device 100, placing the device in imaging mode, which enables it to image the face 2 of the user 1.

Here, in the present preferred embodiment, a guide screen 20 showing an approximated configuration of a general face to be imaged is displayed in the display unit 11 as shown in FIG. 4. The guide screen 20 is configured using dotted lines composed of a plurality of straight lines and curved lines. In addition, the guide screen 20 has a center line 21 drawn in the vertical direction (in the Y direction) to align the center position of the face 2 of the user 1 in the horizontal (left-right) direction and the center position of the image captured by the imaging unit 12 (the center position of the display unit 11) as well as a pair of eye marks 22 drawn to guide the positions (the position in the horizontal direction and the position in the vertical direction) of the right eye and left eye of the user 1 centered on the center line 21 into appropriate positions within the captured images.

Furthermore, the state in which the guide screen 20 is displayed on the display unit 11 is the state immediately prior to actually capturing the face 2 of the user 1 (see FIG. 1) who is to be photographed by the imaging unit 12 as a still image based on the instructions of the control unit 15a (see FIG. 3). Accordingly, the device is configured such that when the user 1 brings the face 2 close to the front (Z2 side) of the imaging unit 12 separated by a specified distance, a preview screen of the face 2 being photographed is displayed in real time on the display unit 11 as shown in FIG. 1. Moreover, the user 1 adjusts their own body posture (the position of the face 2), while looking at the preview screen that is photographing the face 2, to the position in which the guide screen 20 and the image of the face 2 (the face image 30) in the preview screen are superimposed.

Then, it is configured to execute the action of imaging the face 2 of the user 1 at that time when the user 1 touches a specified location within the touch panel portion 11a or presses a specified button key in the operating button key group 18. In addition, the face image 30 is immediately stored in the main memory 15c (see FIG. 3) as image data that captures the face 2. At this time, the date and time information of the capture is also recorded. Note that the main memory 15c constitutes one example of the “storage unit” according to a preferred embodiment of the present invention.

Note that, as shown in FIG. 4, the guide screen 20 displayed on the display unit 11 at the time of capture is configured such that its size and the like can be set according to the individual user 1 (see FIG. 1). Specifically, the position of the pair of eye marks 22 relative to the center line 21 can be moved in the horizontal and vertical directions. The eye marks 22 can also rotate (incline) in the in-plane directions of the guide screen 20 at the same position, and the display size of the eye marks 22 can be adjusted as well. Note that, for reasons of convenience, FIG. 4 shows both the eye marks 22a before rotation (before adjustment) and the eye marks 22b after rotation (after adjustment), but in actuality, there will be only one eye mark 22 on each side, left and right. Furthermore, this sort of guide screen 20 is fine-tuned by the user 1 using a finger or the like to lightly touch (swipe) the portions of the touch panel portion 11a displaying the displayed eye marks 22. Alternatively, it may be configured such that the guide screen 20 is fine-tuned by operating the plus key 18a among the operating button group 18 by pressing it in the up, down, left, and right directions.

Here, in the present preferred embodiment, the information terminal device 100 is configured such that the following sorts of information are provided to the user 1 using the application software described above.

In concrete terms, as shown schematically in FIG. 5, the constitution is such that by comparing the face image 31 captured at a point in time that is in the past relative to the face image 32 captured at the current point in time, which is relatively newer than the past point in time, with these images being in the image data state, the amount of change in color (hue) of the current face image 32 relative to the past face image 31 of the user 1 can be quantitatively ascertained. Moreover, this application software is configured such that the health status of the user 1 (see FIG. 1) is surmised based on the amount of color change from the past face image 31 to the current face image 32, and also such that “health management information” congruent with the surmised health status is displayed on the display unit 11 in a format like that of the message 91 (see FIG. 6).

Note that the past, defined in terms of the current time, may be one day previous or may even be one month previous. It may even be one year previous. When looking for a major change in health status, the face image 31 from one year previous may be compared to the current face image 32; when looking for a subtle change in condition (symptoms), the face image 31 from one month previous (one week previous, one day previous) may be compared to the current face image 32. The application software is configured such that face images 31 from any past point in time can be set for the comparison to the current face image 32.

In addition, as shown in FIG. 5, the present preferred embodiment is configured such that the color change of the entirety of the face image 32 that includes the image 32a (portion of the eyeball area 2a) and the image 32b (portion of the undereye region 2b) captured at the current point in time from the entirety of the face image 31 that includes the image 31a (portion of the eyeball area 2a) and the image 31b (portion of the undereye region 2b) captured at a past point in time is factored into the processing determination when generating the “health management information” rather than ascertaining the simple color change amount from the face image 31 to the face image 32. Furthermore, the constitution is such that after factoring in this color change in the entirety of the current face image 32 from the entirety of the past face image 31, the amount of change in the color (color change information) of the image 32a (portion of the eyeball area 2a) and the image 32b (portion of the undereye region 2b) of the face image 32 relative to the color of the image 31a (portion of the eyeball area 2a) and the image 31b (portion of the undereye region 2b) within the face image 31 is ascertained by the control unit 15a (see FIG. 3). Note that the face image 31, the image 31a, and the image 31b constitute examples of the “image that includes the target diagnostic region captured at a past point in time” according to a preferred embodiment of the present invention. Moreover, the face image 32, the image 32a, and the image 32b constitute examples of the “image that includes the target diagnostic region captured at the current point in time” according to a preferred embodiment of the present invention.

In this case, the control unit 15a performs the control processing which factors in the amount of change (ΔC1) in the skin color of the face 2 of the user 1 in the entirety of the face image 32 captured at the current point in time (face skin color B1) relative to the skin color of the face 2 of the user 1 in the entirety of the face image 31 captured at a past point in time (face skin color A1). Here, some of the factors that can change the skin color of the face 2 between past and current (change from the face skin color A1 to the face skin color B1) might include, for example, skin tanning effects and whitening effects accompanying cosmetics (skin care). That is, it is supposed that, depending on users 1, the skin color of the face 2 might change from white to wheaten, or the degree of its whiteness might be increased by cosmetic whitening.

Accordingly, the present preferred embodiment is configured such that, by comparing, in the data, the entirety of the face image 31 captured at a past point in time with the entirety of the face image 32 captured at the current point in time and also color-corrected to eliminate the effects of color changes in each portion (for example, the forehead 2c, the periphery of the eyeball area 2a, the undereye region 2b, and the chin 2d (see FIG. 1)) arising from changes in the skin color of the face 2 of the user 1 from the past point in time to the current point in time (tanning effects, cosmetic whitening effects, etc.), the control unit 15a ascertains the “net amount of change” in the current color of the image 32a (portion of the eyeball area 2a) and the image 32b (portion of the undereye region 2b) of the face image 32 after color correction relative to the prior color of the image 31a (portion of the eyeball area 2a) and the image 31b (portion of the undereye region 2b) within the face image 31.

Specifically, in FIG. 5, the device is configured to ascertain the amount of change ΔC2 from the pupil and iris color A2 to the pupil and iris color B2 in the eyeball area 2a, the amount of change ΔC3 from the white of the eye color A3 to the white of the eye color B3 in the eyeball area 2a, the amount of change ΔC4 from the undereye skin color A4 to the undereye skin color B4 in the undereye region 2b, and the like, after previously factoring in the amount of change ΔC1 from the face skin color A1 of the entirety of the face image 31 to the face skin color B1 of the entirety of the face image 32. For example, when the color change between the face images 31 and 32 includes tanning effect factors, processing is performed ahead of time to lighten the color of the image overall by the amount of change ΔC1 for the face image 32 (to return it to the pre-tanned state), and thereafter, the amount of color change is ascertained for the various portions (the eyeball area 2a, the undereye region 2b, and the like) between the face image 31 and the face image from which the effects of tanning have been removed. Conversely, in the case of cosmetic whitening, processing is performed ahead of time to instead darken the color of the image overall by the amount of change ΔC1 for the face image 32 (to return it to the pre-whitened state), and thereafter, the amount of color change is ascertained for the various portions (the eyeball area 2a, the undereye region 2b, and the like) between the face image 31 and the face image 32 in which the effects of whitening have not appeared. Note that the color A2 of the pupil and iris, the color A3 of the white of the eye, and the undereye skin color A4 are examples of the “first diagnostic data” according to a preferred embodiment of the present invention. In addition, the color B2 of the pupil and iris, the color B3 of the white of the eye, and the undereye skin color B4 are examples of the “second diagnostic data” according to a preferred embodiment of the present invention.

Thus, it is configured such that, under conditions in which factors involved in changing skin color such as tanning effects and cosmetic whitening effects that do not directly relate to the health management of the user 1 (the amount of change ΔC1) have been eliminated, the application software to be executed in the information terminal device 100 accurately ascertains each of the net amounts of change ΔC2, ΔC3, and ΔC4 in the color of the image 32a (portion of the eyeball area 2a) and the image 32b (portion of the under eye region 2b) captured at the current point in time relative to a past point in time.

Note that for the health management information, health management information pertaining to eyeball health is generated based on diagnostic criteria according to the amount of change ΔC2 from the pupil and iris color A2 to the pupil and iris color B2 in the eyeball area 2a, while separate health management information pertaining to eyeball health is generated based on diagnostic criteria according to the amount of change ΔC3 from the white of the eye color A3 to the white of the eye color B3 in the eyeball area 2a. It is also configured to separately generate health management information pertaining to the health of the various parts of the human body (organs and the like) that are related to skin color change based on diagnostic criteria according to the amount of change ΔC4 from the undereye skin color A4 to the undereye skin color B4 in the undereye region 2b.

Furthermore, in the present preferred embodiment, when calculating the amount of skin color change ΔC1 due to tanning effects, cosmetic whitening effects, and the like, which do not directly relate to health management of the user 1, the following sort of image data processing is applied. Specifically, it is configured such that the amount of skin color change ΔC1 due to tanning effects, cosmetic whitening effects, and the like is calculated based on the entirety of the face image 31 that includes the image 31a (portion of the eyeball area 2a) and the image 31b (portion of the undereye region 2b) at a past point in time used as an achromatic image (the grayscale image including the white, black, and their intermediate colors (grays), from which color components are removed) which is produced as a result of the image processing by the control unit 15a and the entirety of the face image 32 that includes the image 32a (portion of the eyeball area 2a) and the image 32b (portion of the undereye region 2b) at the current point in time used as an achromatic image (grayscale image) which is produced as a result of the image processing by the control unit 15a. Note that comparison between such achromatic images is a process run on image data, and no achromatic images are actually displayed on the display unit 11.

Moreover, the present preferred embodiment is configured to generate health management information with content that is congruent with the amount of color change (for example, the message 91 (see FIG. 6)) only in cases where the color change amounts of individual portions (net change amounts) exceed specified threshold values when the colors A2, A3, and A4 of the image 31a (portion of the eyeball area 2a) and the image 31b (portion of the undereye region 2b) within the face image 31 are compared to the colors B2, B3, and B4 of the image 32a (portion of the eyeball area 2a) and the image 32b (portion of the undereye region 2b) of the post-correction face image 32 from which tanning effects and the like have been eliminated. Conversely, it is configured to not generate health management information in cases where it is determined that the ascertained amounts of color change do not meet the specified threshold values.

In addition, the present preferred embodiment is configured to ascertain the respective amounts of change ΔC2, ΔC3, and ΔC4 in the colors present in each of the images 32a and 32b by respectively comparing reds, greens, and blues to each other between the individual color scale values corresponding to the three primary colors of light (red color scale values, green color scale values, and blue color scale values) in the image 31a (31b) of the face image 31 (past) and the individual color scale values corresponding to the three primary colors of light (red color scale values, green color scale values, and blue color scale values) in the image 32a (32b) of the face image 32 (current) when the colors A2, A3, and A4 of the image 31a (portion of the eyeball area 2a) and the image 31b (portion of the undereye region 2b) within the face image 31 are compared to the colors B2, B3, and B4 of the image 32a (portion of the eyeball area 2a) and the image 32b (portion of the undereye region 2b) of the post-correction face image 32 from which the effects of tanning and the like have been eliminated. It is also configured such that, when comparing within the individual color scale values (red color scale values, green color scale values, and blue color scale values), it uses in the operations at the time of comparison the respective average values of the red color scale values, green color scale values, and blue color scale values of the plurality of pixels (individual pixels) included in the image 31a (31b) of the face image 31 captured in the past and the respective average values of the red color scale values, green color scale values, and blue color scale values of the plurality of pixels (individual pixels) included in the image 32a (32b) of the face image 32 captured at the current point in time.

Furthermore, there are a pair of images 31a (portions of the eyeball areas 2a) of the face image 31 on the left and right (the image 31a of the eyeball area 2a of the right eye and the image 31a of the eyeball area 2a of the left eye) as shown in FIG. 5, and the present preferred embodiment is configured such that the pupil and iris color A2 in the eyeball area 2a (red color scale values, green color scale values, and blue color scale values) is ascertained using the average value for the left and right images 31a. Moreover, the constitution is such that the white of the eye color A3 (red color scale values, green color scale values, and blue color scale values) is also ascertained using the average value for the left and right images 31a. Similarly, there are also a pair of images 31b (portions of the undereye regions 2b) of the left and right, and the constitution is such that the undereye skin color A4 (red color scale values, green color scale values, and blue color scale values) in the undereye region 2b is ascertained using the average value for the left and right images 31b. In addition, with regard to these features, the same also applies to the face image 32 captured at the current point in time.

The information terminal device 100 applies image data processing using this sort of technique to quantitatively ascertain the amounts of color change in the current face image 32 relative to the past face image 31 of the user 1 and surmises the health status of the user 1 based on these color change amounts. Furthermore, the constitution is such that “health management information” in accordance with the surmised health status is displayed on the display unit 11 as the message 91 (see FIG. 6).

Moreover, the information terminal device 100 is configured such that the following sorts of functions can also be output in addition to the aforementioned image data processing for the captured face images 30 (the past face image 31 and the current face image 32).

In concrete terms, the constitution is such that when “health management information” is generated based on the result of ascertaining the amount of color change (the amount of net color change) in the current face image 32 of the user 1 relative to the past face image 31, pigmented spots (freckles, birthmarks, and the like) or tumorous areas (eczema, boils (pimples), moles, and the like) 51 (see FIG. 7) appeared on the face 2 at the current point in time that were not present at a past point in time can be identified in the captured face image 31 of the user 1, which includes the image 31a (portion of the eyeball area 2a) and the image 31b (portion of the undereye region 2b). That is, the device is configured to not only generate health management information based on the amount of change in simple color from the past face image 31 to the current face image 32, but also generate health management information that factors in information on identified pigmented spots or tumorous areas. Accordingly, it is configured such that the health management information displayed on the display unit 11 includes realistic (practical) health management information for the user 1.

In addition, the present preferred embodiment is configured such that image data processing via the following technique is applied when identifying pigmented spots or tumorous areas 51 that were not present at a past point in time but are preset on the face 2 of the user 1 at the current point in time.

Specifically, as shown schematically in FIG. 7, it is configured to create a composite image (image data) 37 that superimposes a first inverted image (image data) 35, which inverts the white and black portions of the entirety of the face image 31 that includes the image 31a (portion of the eyeball area 2a) and the image 31b (portion of the undereye region 2b) taken at a past point in time and converted into an achromatic image (grayscale image) from the color image state immediately after capture, and a second inverted image (image data) 36, which inverts the white and black portions of the entirety of the face image 32 that includes the image 32a (portion of the eyeball area 2a) and the image 32b (portion of the undereye region 2b) at the current point in time, and to have the control unit 15a (see FIG. 3) perform control to determine whether or not pigmented spots or tumorous areas 51 have been produced on the face 2 of the user 1 (see FIG. 1) in this composite image (image data) 37. Note that in the present preferred embodiment, the second inverted image 36 is created based on image data which has been color-corrected by previously factoring in the amount of change ΔC1 from the face skin color A1 of the entirety of the face image 31 in the color image to the face skin color B1 of the entirety of the face image 32 as described above.

Furthermore, in the creation of the composite image 37, it is configured to perform the image data processing which superimposes the first inverted image 35 and the second inverted image 36 in a state in which their brightness (luminance) is reduced by approximately 50% each. Accordingly, the regions that have not produced pigmented spots or tumorous areas 51 within the composite image 37 appear as a uniform gray of the 128th gradation among the 256 gradations, while regions that have produced pigmented spots or tumorous areas 51 are recognized as regions that have color data other than the gray of the 128th gradation. Note that such data creation processing for the first inverted image 35 and the second inverted image 36 and data creation processing for the composite image 37 that superimposes the first inverted image 35 and the second inverted image 36 is all processing within the image data. Thus, the constitution is such that pigmented spots and tumorous areas 51 newly present on the face 2 of the user 1 can be easily and precisely identified.

Moreover, the present preferred embodiment is configured such that the ambient type of environmental light of the information terminal device 100 (see FIG. 1) can be input when the user 1 (see FIG. 1) captures their own face 2 using the imaging unit 12 (see FIG. 1). Specifically, a settings screen 60 like that shown in FIG. 8 is displayed on the display unit 11 when the user 1 touches a specified location within the touch panel portion 11a or presses a specified button key in the operating button key group 18. A plurality of selectable types of environmental light are set up in the settings screen 60. Then, the constitution is such that the user 1 can operate the touch panel portion 11a or the plus key 18a to set the type of environment light at the time of imaging.

Accordingly, it is configured to capture the face image 30 (see FIG. 1) in the state in which color has been corrected for the preset type of environmental light when capturing the face 2 of the user 1 (see FIG. 1) using the imaging unit 12. This color correction processing is preferably processing that is enabled in cases where an environmental light type has been set according to the ambient environment (brightness) of the information terminal device 100 when the user 1 captures the face image 31 (see FIG. 5) by imaging the face 2 at a past point in time or when the user 1 captures the face image 32 (see FIG. 5) by imaging the face 2 at the current point in time as well. As a result, the constitution is such that the conditions for environmental light at the time of imaging (imaging conditions) at individual points in time are matched to the same status in the entirety of the past and current face images 30 (past face image 31 and current face image 32) that are compared to each other.

In addition, as shown in FIG. 2, the device is configured such that the illuminance sensor 13 is used to constantly detect the brightness of the ambient environmental light of the information terminal device 100. Consequently, it is configured such that when the environmental light (brightness) is determined to be too low (too dark) based on the detection results of the illuminance sensor 13, a message such as “please increase the brightness” is displayed on the display unit 11. Conversely, it is configured such that when the environmental light is determined to be too high (too bright), a message such as “please decrease the brightness a little” is displayed on the display unit 11 (see FIG. 2). The information terminal device 100 is thus configured to prevent imaging errors caused by environmental light.

Thus, the information terminal device 100 (see FIG. 1) equipped with application software that has health management functions according to the present preferred embodiment is provided.

Next, the control processing flow of the control unit 15a when it executes application software that has health management functions in the information terminal device 100 according to the present preferred embodiment will be described with reference to FIG. 1, FIG. 3, and FIGS. 4 through 9.

As shown in FIG. 9, the control unit 15a (see FIG. 3) first determines in step S1 whether or not the user 1 (see FIG. 1) has performed the specified operation to start application software that has health management functions (a health management application), and it repeats this processing until it determines that the specified operation to start the application software has been performed. In step S1, the response is considered to be YES when it is determined that the user 1 has touched a specified location within the touch panel portion 11a (see FIG. 1) or pressed the specified button key in the operating button group 18 (see FIG. 1).

If it is determined in step S1 that a specified operation for starting the application software has been performed, the imaging unit 12 (see FIG. 1) is driven in step S2. Then, in step S3, the guide screen 20 (see FIG. 4) is displayed on the display unit 11 (see FIG. 1). Accordingly, as a result of the user 1 bringing the face 2 closer to the front (Z2 side) of the imaging unit 12 separated by a specified distance, a preview screen of the face 2 being photographed is displayed in real time on the display unit 11 as shown in FIG. 1.

Subsequently, it is determined in step S4 whether or not the user 1 has performed an operation equivalent to pressing a shutter button, and this processing is repeated until it is determined that an operation equivalent to pressing the shutter button has been performed. Then, if it is determined in step S4 that an operation equivalent to pressing the shutter button has been performed, then the imaging unit 12 is driven to perform the actual imaging in step S5. As a result, the face image 30 that images the face 2 of the user 1 at that time (the current face image 32 (see FIG. 5)) is captured. Thereafter, in step S6, the data of the face image 30 (the current face image 32) is stored in the main memory 15c (see FIG. 3).

Afterward, in step S7, the control unit 15a (see FIG. 3) determines whether or not the data of a face image 31 (see FIG. 5) captured at a past point in time is stored in the main memory 15c (see FIG. 3), and if it is determined that the data of a face image 31 captured at a past point in time is not stored in the main memory 15c, then this control procedure terminates. That is, driving of the imaging unit 12 by the control unit 15a is halted, and the application software terminates.

Furthermore, if it is determined in step S7 that the data of a face image 31 captured at a past point in time (see FIG. 5) is stored in the main memory 15c, then, in step S8, the color information contained in this data of the face image 31 captured at a past point in time is acquired by the control unit 15a (see FIG. 3). In such cases, as shown in FIG. 5, the face skin color A1 of the entirety of the face image 31, the pupil and iris color A2 in the eyeball area 2a, the white of the eye color A3 in the eyeball area 2a, and the undereye skin color A4 in the undereye region 2b contained in the data of the face image 31 are acquired by the control unit 15a (see FIG. 3).

Moreover, in step S9, the color information contained in the data of the face image 32 just imaged and stored in the main memory 15c is acquired by the control unit 15a (see FIG. 3). In this case, as shown in FIG. 5, the face skin color B1 of the entirety of the face image 32, the pupil and iris color B2 in the eyeball area 2a, the white of the eye color B3 in the eyeball area 2a, and the undereye skin color B4 in the undereye region 2b contained in the data of the face image 32 are acquired by the control unit 15a (see FIG. 3).

In addition, in the present preferred embodiment, in step S10, the control unit 15a ascertains the amount of change in the current color (the pupil and iris color B2, the white of the eye color B3, or the undereye skin color B4) of the image 32a (portion of the eyeball area 2a) and the image 32b (portion of the undereye region 2b) of the color-corrected face image 32 relative to the prior color (the pupil and iris color A2, the white of the eye color A3, or the undereye skin color A4) of the image 31a (portion of the eyeball area 2a) and the image 31b (portion of the undereye region 2b) within the face image 31. Specifically, in FIG. 5, the amount of change ΔC2 from the pupil and iris color A2 to the pupil and iris color B2 in the eyeball area 2a, the amount of change ΔC3 from the white of the eye color A3 to the white of the eye color B3 in the eyeball area 2a, the amount of change ΔC4 from the undereye skin color A4 to the undereye skin color B4 in the undereye region 2b, or the like is ascertained after previously factoring in the amount of change ΔC1 from the face skin color A1 of the entirety of the face image 31 to the face skin color B1 of the entirety of the face image 32.

Then, health management information congruent with the color change amounts calculated by the control unit 15a in step S10 is generated in step S11. In this case, health management information pertaining to eyeball health is generated based on diagnostic criteria according to the amount of change ΔC2 from the pupil and iris color A2 to the pupil and iris color B2 in the eyeball area 2a, and separate health management information pertaining to eyeball health is also generated based on diagnostic criteria according to the amount of change ΔC3 from the white of the eye color A3 to the white of the eye color B3 in the eyeball area 2a. Furthermore, health management information pertaining to the health of the various portions of the human body (organs and the like) that are related to skin color change is generated based on diagnostic criteria according to the amount of change ΔC4 from the undereye skin color A4 to the undereye skin color B4 in the undereye region 2b.

Note that, in step S10, an operation processing is performed which not only generates health management information based on the simple amount of change in color from the past face image 31 of the user 1 to the current face image 32 (the amount of net color change), but which also recognizes pigmented spots (freckles, birthmarks, and the like) or tumorous areas (eczema, boils (pimples), moles, and the like) 51 that are present on the face 2 (see FIG. 1) at the current point in time but were not present at a past point in time in the face image 32 that includes the image 32a (portion of the eyeball area 2a) and the image 32b (portion of the undereye region 2b) of the user 1 as shown in FIG. 7. Specifically, the control unit 15a (see FIG. 3) internally creates a composite image 37 by superimposing the first inverted image 35 that inverts the white and black portions of the entirety of the face image 31 and the second inverted image 36 that inverts the white and black portions of the entirety of the face image 32 which have been data-converted into achromatic images, and the control unit 15a determines whether or not pigmented spots or tumorous areas 51 exist in the composite image 37. Accordingly, if the presence of pigmented spots or tumorous areas 51 is recognized as a result of operation processing, information about this subject is also appended to the health management information in step S11.

Then, in step S12, the health management information generated in step S11 (for example, the message 91 (see FIG. 6) or the like) is displayed on the display unit 11 (see FIG. 6). Thus, this control procedure terminates.

In the present preferred embodiment, as was described above, the control unit 15a is provided which detects the amounts of color change (ΔC2, ΔC3, and ΔC4) of the pupil and iris color B2 (the white of the eye color B3 or the undereye skin color B4 of the undereye region 2b) at the current point in time relative to the pupil and iris color A2 (the white of the eye color A3 or the undereye skin color A4 of the undereye region 2b) at a past point in time stored in the main memory 15c after factoring in the color change (ΔC1) to the face skin color B1 of the entirety of the face image 32 that includes the image 32a (portion of the eyeball area 2a) and the image 32b (portion of the undereye region 2b) captured at the current point in time from the face skin color A1 of the entirety of the face image 31 that includes the image 31a (portion of the eyeball area 2a) and the image 31b (portion of the undereye region 2b) captured at a past point in time by the imaging unit 12. Consequently, the amount of color change in the eyeball area 2a (or the undereye region 2b) becomes the amount of color change after factoring in color change from the past (the face image 31) to the current (the face image 32) of the face images 30 in their entirety, which include the eyeball area 2a and the undereye region 2b. Therefore, the amount of color change in the eyeball area 2a or the undereye region 2b described above can be detected based on not only color changes caused by factors that are directly related to the condition, symptoms, and the like of the user 1 but also color changes originated from other factors such as skin tanning and whitening due to cosmetics (skin care). Accordingly, accuracy and precision are increased when generating health management information (the message 91) and the like based on the amount of color change in the eyeball area 2a or the undereye region 2b, so the user 1 can use this information terminal device 100 to accurately perform self-diagnosis involving health management.

Moreover, the present preferred embodiment is configured to generate health management information (the message 91) for the user 1 based on the amount of color change detected by the control unit 15a and display it on the display unit 11. This makes it possible for the user 1 to easily perform self-diagnosis involving health management based on health management information (the message 91) displayed on the display unit 11.

In addition, in the present preferred embodiment, the message 91 displayed on the display unit 11 includes the determination result from determining the health status of the user 1 based on the amount of color change in the face image 30 (operation processing result) from the control unit 15a. This enables the user 1 to obtain a more accurate and precise diagnosis of health status using the information terminal device 100 because it adds an evaluation of health status by the control unit 15a based on the health management information (the message 91) to the self-diagnosis by the user 1.

Furthermore, in the present preferred embodiment, the control unit 15a is configured to perform control that generates the health management information (the message 91) of the user 1 based on the results of detecting (ascertaining) the amount of color change in the image 32a (32b) at the current point in time relative to the image 31a (31b) at a past point in time by factoring in the change of the skin color of the face 2 of the user 1 in the entirety of the face image 32 which includes the image 32a (portion of the eyeball area 2a) and the image 32b (portion of the undereye region 2b) captured at the current point in time compared to the entirety of the face image 31 which includes the image 31a (portion of the eyeball area 2a) and the image 31b (portion of the undereye region 2b) captured at a past point in time (based on the results of detecting the respective amounts of color change of the pupil and iris color B2, the white of the eye color B3, or the undereye skin color B4 at the current point in time compared to the pupil and iris color A2, the white of the eye color A3, or the undereye skin color A4 of the undereye region 2b at a past point in time). This enables health management information (the message 91) to be generated based on the amount of color change from the images 31a to 32a (from the images 31b to 32b) that is detected (ascertained) after factoring in change in skin color of the face 2 from past (the face image 31) to current (the face image 32) in the entirety of the face image 30 which includes the images 30a and 30b. To with, various factors involved in changing the skin color of the face 2 of the user 1 can greatly contribute to changes in the color of the image 30a (portion of the eyeball area 2a) and the image 30b (portion of the undereye region 2b), so it is possible to generate health management information (the message 91) that is more accurate and precise because it factors in changes in the skin color of the face 2.

Moreover, in the present preferred embodiment, the control unit 15a is programmed so as to detect (ascertain) the amount of color change of the eyeball area 2a and the undereye region 2b at the current point in time relative to the eyeball area 2a and the undereye region 2b at a past point in time by comparing the face image 31 which includes the image 31a (portion of the eyeball area 2a) and the image 31b (portion of the undereye region 2b) captured at a past point in time and the face image 32 which includes the image 32a (portion of the eyeball area 2a) and the image 32b (portion of the undereye region 2b) that is captured at the current point in time and corrected to remove the effects of color changes in the eyeball area 2a and the undereye region 2b caused by changes in the skin color of the face 2 of the user 1 from the past point in time to the current point in time (the amount of change ΔC1 from the face skin color A1 to the face skin color B1) (the amounts of color change ΔC2, ΔC3, and ΔC4 of the pupil and iris color B2, the white of the eye color B3, or the undereye skin color B4 at the current point in time relative to the pupil and iris color A2, the white of the eye color A3, or the undereye skin color A4 of the undereye region 2b at a past point in time). As a result, various factors such as skin tanning effects or whitening effects accompanying cosmetics (skin care) are eliminated in advance from factors that can cause changes in skin color over the entirety of the face image 30 that includes the eyeball area 2a and undereye region 2b whose past and current images are to be compared. Therefore, it is possible to accurately ascertain amounts of color change in the eyeball area 2a and undereye region 2b at the current point in time relative to a past point in time under conditions that exclude factors affecting skin color change such as tanning or cosmetic whitening not directly involved in the health management of the user 1 (net color change amount). As a result, it is possible to easily generate health management information (the message 91) that enables accurate self-diagnosis.

In addition, in the present preferred embodiment, the control unit 15a is configured to calculate the amount of color change ΔC1 in the skin of the face 2 of the user 1 in the entirety of the face image 30 including the eyeball area 2a and the undereye region 2b based on the entirety of the face image 31 as an achromatic image (grayscale image) including the image 31a (portion of the eyeball area 2a) and the image 31b (portion of the undereye region 2b) at a past point in time and the entirety of the face image 32 as an achromatic image (grayscale image) including the image 32a (portion of the eyeball area 2a) and the image 32b (portion of the undereye region 2b) at the current point in time. Consequently, the amount of color change ΔC1 in the skin of the face 2 of the user 1 can be easily calculated based on the brightness (darkness) of the entirety of the image composed of achromatic colors including white, black, and their intermediate colors (grays), from which color components have been removed. Furthermore, because the image processing performed by the control unit 15a involves handling of achromatic image data, the processing load on the control unit 15a (the information terminal device 100) can be significantly reduced compared to handling of color image data.

Moreover, in the present preferred embodiment, the control unit 15a is configured to generate health management information (the message 91) congruent with the amounts of color change ΔC2, ΔC3, and ΔC4 of the eyeball area 2a and the undereye region 2b when the amounts of color change in the eyeball area 2a (the pupil and iris color B2 or the white of the eye color B3) and the undereye region 2b (the undereye skin color B4) at the current point in time relative to the eyeball area 2a (the pupil and iris color A2 or the white of the eye color A3) and the undereye region 2b (the undereye skin color A4) at a past point in time exceed specified thresholds. Consequently, health management information (the message 91) is generated only when the amounts of color change in the eyeball area 2a and the undereye region 2b exceed a threshold, and no health management information is generated when the amounts of color change in the eyeball area 2a and the undereye region 2b do not meet the threshold. That is, it is possible to generate only health management information which is genuinely necessary for the color change amounts ΔC2, ΔC3, and ΔC4 of the eyeball area 2a and undereye region 2b that cannot be ignored, without being excessively sensitive to color change amounts in the eyeball area 2a and undereye region 2b that can normally be ignored (generating erroneous health management information), so more accurate and precise health management information is provided to the user 1.

In addition, in the present preferred embodiment, the control unit 15a is programmed to detect (ascertain) the amounts of change ΔC2, ΔC3, and ΔC4 of the colors of the eyeball area 2a and the undereye region 2b by respectively comparing reds, greens, and blues to each other between the individual color scale values corresponding to the three primary colors of light (red color scale values, green color scale values, and blue color scale values) in the eyeball area 2a and the undereye region 2b captured at a past point in time and the individual color scale values corresponding to the three primary colors of light (red color scale values, green color scale values, and blue color scale values) in the eyeball area 2a and the undereye region 2b captured at the current point in time. As a result, the amounts of color change ΔC2, ΔC3, and ΔC4 of the image 30a and the image 30b of the eyeball area 2a and undereye region 2b can be detected (ascertained) by using three amounts of change as indexes, i.e., amount of red change, amount of green change, and amount of blue change, corresponding to the three primary colors of light in the eyeball area 2a and the undereye region 2b between past and current. That is, such color change amounts can be easily ascertained in the image processing that is performed by the control unit 15a (the information terminal device 100).

Furthermore, in the present preferred embodiment, the control unit 15a is programmed to detect (ascertain) the amounts of change ΔC2, ΔC3, and ΔC4 of the colors of the eyeball area 2a and the undereye region 2b by comparing the respective average values of the individual color scale values corresponding to the three primary colors of light (red color scale values, green color scale values, and blue color scale values) in the eyeball area 2a (the image 31a) and the undereye region 2b (the image 31b) captured at a past point in time and the respective average values of the individual color scale values corresponding to the three primary colors of light (red color scale values, green color scale values, and blue color scale values) in the eyeball area 2a (the image 32a) and the undereye region 2b (the image 32b) captured at the current point in time, for each of the colors (red, green, and blue). Consequently, the amount of data used in comparison between past and current images can be decreased by using the respective average values for the individual color scale values of the individual pixels compared to the case of ascertaining the amount of change in individual color scale values (red, green, and blue) in units of the individual pixels that make up the images that capture the eyeball area 2a and the undereye region 2b (the image 30a and the image 30b). Accordingly, the processing load on the control unit 15a (information terminal device 100) is reduced significantly, and processing is also performed quickly.

Moreover, the present preferred embodiment is configured such that pigmented spots (freckles, birthmarks, and the like) or tumorous areas (eczema, boils (pimples), moles, and the like) present on the face 2 at the current point in time that were not present at a past point in time are identified by the control unit 15a in the face image 30 which includes the image 30a (portion of the eyeball area 2a) and the image 30b (portion of the undereye region 2b). In addition, the control unit 15a is configured to generate the health management information (the message 91) of the living body by factoring in the information on the identified pigmented spots or tumorous areas. As a result, not only are the amounts of simple color change in the eyeball area 2a and the undereye region 2b (the image 30a and the image 30b) made available as a basis of determination for generating health management information, but health management information (the message 91) can also be generated after simultaneous detection (identification) of newly present pigmented spots or tumorous areas in the living body from the amount of color change, so it is possible to provide the user 1 with more realistic (practical) health management information germane to the health management of the user 1.

Furthermore, in the present preferred embodiment, the face image 30 which includes the image 30a (portion of the eyeball area 2a) and the image 30b (portion of the undereye region 2b) captured by the imaging unit 12 is a color image, and the control unit 15a is configured to detect appearance of pigmented spots or tumorous areas 51 in the face 2 of the user 1 based on a composite image (image data) 37 that superimposes a first inverted image (image data) 35 that inverts the white and black portions of the entirety of the face image 31 which includes the image 31a (portion of the eyeball area 2a) and the image 31b (portion of the undereye region 2b) at a past point in time and that has been converted from a color image to an achromatic image (gray scale image) and a second inverted image (image data) 36 that inverts the white and black portions of the entirety of the face image 32 which includes the image 32a (portion of the eyeball area 2a) and the image 32b (portion of the undereye region 2b) at the current point in time. As a result, the pigmented spots or tumorous areas 51 newly present on the living body can be easily and precisely identified in image processing by the control unit 15a that uses the composite image 37 that superimposes the first inverted image 35 and the second inverted image 36.

Moreover, the present preferred embodiment is configured such that it is possible to input the type of environmental light when the imaging unit 12 is used to image the face image 30 which includes the eyeball area 2a and the undereye region 2b, and the control unit 15a is configured to detect the amounts of color change ΔC2, ΔC3, and ΔC4 of the eyeball area 2a and the undereye region 2b by comparing the face image 31 which includes the eyeball area 2a and the undereye region 2b captured at a past point in time and the face image 32 which includes the eyeball area 2a and the undereye region 2b captured at the current point in time after performing color correction on the face image 31 which includes the image 31a (portion of the eyeball area 2a) and the image 31b (portion of the undereye region 2b) captured at a past point in time and/or the face image 32 which includes the image 32a (portion of the eyeball area 2a) and the image 32b (portion of the undereye region 2b) captured at the current point in time based on the type of environmental light that is input at each point in time. As a result, the conditions pertaining to environmental light at the time of imaging (imaging conditions) at individual points in time can be matched to the same status in the entirety of the face image 30 which includes the eyeball area 2a and the undereye region 2b, for which past and current images are compared to each other. Accordingly, it is possible to accurately ascertain the amounts of color change ΔC2, ΔC3, and ΔC4 of the eyeball area 2a and the undereye region 2b at the current point in time from the eyeball area 2a and the undereye region 2b at a past point in time.

Note that the preferred embodiments disclosed herein merely constitute illustrative examples in all respects and should be considered to be nonrestrictive. The scope of the present invention is indicated not by the description of the aforementioned preferred embodiments but rather by the scope of the claims, and it includes all modifications within the scope of the patent claims.

For example, in various preferred embodiments of the present invention, an example was shown in which the face 2 (portions of the left and right eyeball areas 2a and the portions of the undereye regions 2b) of the user 1 is preferably used as the “target diagnostic region”. However, the present invention is not limited to this. For instance, it may also be configured to generate the health management information for the user 1 by capturing images of a hand, leg, abdomen, chest area, back portion, or the like as the target diagnostic region rather than the face 2. Furthermore, the target diagnostic region on the face 2 may also be a region such as the nose area (tip or base), lips, tongue, mouth, or the like besides the eyeball area. Moreover, various preferred embodiments of the present invention may also be applied to the identification of wrinkles (laugh lines) due to aging of skin in addition to pigmented spots.

In addition, various preferred embodiments of the present invention showed changes in skin color related to factors such as skin tanning effects and whitening effects accompanying cosmetics (skin care) as examples of skin color changes from the entirety of the face image 31 captured at a past point in time to the entirety of the face image 32 captured at the current point in time; however, the present invention is not limited to this. For example, even in cases such as the absorption of alcohol or the like within the body turning the skin red or daily (periodic) administration of medicines and the like causing the skin color to change, the amounts of color change in the “target diagnostic region” can be accurately ascertained in a state in which the effects of such changes in skin color are removed by applying the present invention.

Furthermore, in various preferred embodiments of the present invention, an example was shown in which the control unit 15a preferably is programmed to perform control that ascertains the amounts of change of the colors of the eyeball area 2a and the undereye region 2b by comparing the respective average values of the individual color scale values corresponding to the three primary colors of light (red color scale values, green color scale values, and blue color scale values) in the eyeball area 2a (the image 31a) and the undereye region 2b (the image 31b) captured at a past point in time and the respective average values of the individual color scale values corresponding to the three primary colors of light (red color scale values, green color scale values, and blue color scale values) in the eyeball area 2a (the image 32a) and the undereye region 2b (the image 32b) captured at the current point in time, for each of the colors (red, green, and blue), but the present invention is not limited to this. For instance, the captured images may also be compared to each other for each color (red, green, and blue) by another method without finding the average values with the captured image (pixels) in each of the color scale values.

Moreover, in various preferred embodiments of the present invention, an example was shown which preferably uses a value that averages the color of the left eyeball area and the right eyeball area of the image 31a (portion of the eyeball area 2a) and which uses a value that averages the color of the left undereye region and the right under eye region of the image 31b (portion of the under eye region 2b). However, the present invention is not limited to this. For example, instead of calculating average values for the right-side portions and the left-side portions in this manner, it would also be possible to individually ascertain the amount of color change between past and current regarding the right eyeball area (right undereye region) and the amount of color change between past and current regarding the left eyeball area (left undereye region). Doing so allows the target diagnostic region of the living body for which the generated health management information is effective to be defined more precisely, so the health management information will be more beneficial for the user as well.

In addition, in various preferred embodiments of the present invention, an example was shown which preferably is configured to ascertain color change amounts using individual color scale values (R: red color scale values, G: green color scale values, and B: blue color scale values) corresponding to the three primary colors of light. However, the present invention is not limited to this. Systems for quantitatively evaluating color data other than RGB color scale values, such as the subtractive color system CMY(K) or the YUV system, which is composed of brightness signals and color difference signals, may also be used to quantify color data.

Furthermore, in various preferred embodiments of the present invention, an example was shown which is preferably configured to notify the user 1 of health management information by displaying the message 91 on the display unit 11, but the present invention is not limited to this. For example, it would also be possible to configure the device so as to convert the message 91 to audio data and then to provide audio output through the speaker 17, thus notifying the user 1 of health management information.

Moreover, in various preferred embodiments of the present invention, an example was shown in which the guide screen 20 showing an approximated configuration of a general face preferably is displayed on the display unit 11 to guide the posture and attitude of the face 2 of the user 1 during imaging, but the present invention is not limited to this. The device may also be configured to recognize the individual elements (eyebrows, eyes, nose, mouth, etc.) of the face 2 of the user 1 with the use of image recognition technology and to output sound for guidance from the speaker 17 based on these recognition results, thus guiding the posture and attitude of the face 2 of the user 1 during imaging.

In addition, in various preferred embodiments of the present invention, an example was shown which is configured such that when the environmental light (brightness) is determined to be too low (too dark) based on the detection results of the illuminance sensor 13, a message such as “please increase the brightness” preferably is displayed on the display unit 11. However, the present invention is not limited to this. A light source portion such as an LED may be provided on the information terminal device 100 and configured to emit light from the light source portion to supplement the amount of light during imaging when environmental light is insufficient. In this case, it is preferable that the amount of light of the light source portion be made adjustable depending on the extent of insufficiency in the amount of light by coordinating the control with the illuminance sensor 13. Providing a light source that can adjust the amount of light makes it possible to keep the amount of light fairly constant during imaging, so images (the entirety of the image that includes the target diagnostic region) are obtained with the quality thereof being kept stable from one imaging to the next.

Furthermore, in various preferred embodiments of the present invention, an example involving imaging a human body (the user 1) was shown, but the present invention is not limited to this. The present invention can also be applied to a case in which animals (living bodies), other than human bodies, including pets such as cats and dogs as well as dogs, cats, monkeys, mice, and the like raised for laboratory purposes, are imaged in order to manage the health of these living bodies.

Moreover, in various preferred embodiments of the present invention, examples were shown in which simple relative comparisons preferably are made between the face image 31 captured at a past point in time and the face image 32 captured at the current point in time. However, the present invention is not limited to this. Specifically, the device may also be configured such that the result of comparison between a face image 31 of one year prior and the current face image 32, the result of comparison between a face image 31 of one month prior and the current face image 32, the result of comparison between a face image 31 of one week prior and the current face image 32, and the result of comparison between a face image 31 of one day prior and the current face image 32 are sequentially stored in the main memory 15c, and the “health management information” is then generated based on data that graphs color changes (trends) between each result. In addition, the constitution may also be such that comparison results of face images between new and old to each other made in the past are compiled sequentially in the main memory 15c, and after ascertaining shifts in health status, the “health management information” is then generated. There are no particular restrictions with regard to this point.

Furthermore, in various preferred embodiments of the present invention, because of explanatory convenience, the control procedure of the control unit 15a of the information terminal device 100 was described using a flow-driven type of flowchart that performs processing sequentially according to a processing flow. However, the present invention is not limited to this. In the present invention, the control process of the control unit 15a may be accomplished by event-driven type of processing that executes processes in event units. In such cases, processing may be accomplished by completely event-driven processes or by a combination of event-driven and flow-driven processes.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. An information terminal device comprising:
an imaging unit which captures images that include a target diagnostic region in a living body;
a diagnostic data extraction unit which extracts diagnostic data for the target diagnostic region from the images captured by the imaging unit;
a storage unit which stores the images captured by the imaging unit and the diagnostic data extracted by the diagnostic data extraction unit; and
a detecting unit which detects an amount of color change in the target diagnostic region from first diagnostic data and second diagnostic data newer than the first diagnostic data that are stored in the storage unit after accounting for color changes in an entire image that includes the target diagnostic region captured by the imaging unit at a current point in time compared to an entire image that includes the target diagnostic region captured at a past point in time.

2. The information terminal device according to claim 1, further comprising a generating unit which generates health management information for the living body based on the amount of color change detected by the detecting unit.

3. The information terminal device according to claim 2, further comprising a determining unit which determines health status based on the health management information.

4. The information terminal device according to claim 2, wherein the generating unit is configured to generate health management information of the living body based on results of detection by the detecting unit of amounts of change in color in the target diagnostic region in the second diagnostic data relative to the first diagnostic data after factoring in color changes in the skin of the living body in the entire image that includes the target diagnostic region captured at the current point in time compared to the entire image that includes the target diagnostic region captured at a past point in time.

5. The information terminal device according to claim 4, wherein the detecting unit is configured to detect amounts of color change in the target diagnostic region in the second diagnostic data relative to the first diagnostic data by comparing an image that includes the target diagnostic region captured at a past point in time with an image that includes the target diagnostic region captured at the current point in time and corrected to exclude effects of color change in the target diagnostic region arising from changes in skin color from the past point in time to the current point in time.

6. The information terminal device according to claim 4, wherein the device is configured to calculate the amount of color change of the skin of the living body in the entire image that includes the target diagnostic region based on the entire image as an achromatic image that includes the target diagnostic region at a past point in time and the entire image as an achromatic image that includes the target diagnostic region at the current point in time.

7. The information terminal device according to claim 2, wherein the generating unit is configured to generate the health management information according to amounts of change in the color of the target diagnostic region when the amount of color change in the target diagnostic region in the second diagnostic data relative to the first diagnostic data exceeds a specified threshold value.

8. The information terminal device according to claim 2, wherein the detecting unit is configured to detect amounts of change in color in the target diagnostic region of the second diagnostic data relative to the first diagnostic data by comparing individual color scale values corresponding to three primary colors of light at the target diagnostic region captured at a past point in time and the individual color scale values corresponding to the three primary colors of light at the target diagnostic region captured at the current point in time, for each of the three primary colors.

9. The information terminal device according to claim 8, wherein the information terminal device is configured such that the amounts of change in color in the target diagnostic region of the second diagnostic data relative to the first diagnostic data are detected by the detecting unit by comparing respective average values for the individual color scale values corresponding to the three primary colors of light at the target diagnostic region captured at a past point in time and the respective average values for the individual color scale values corresponding to the three primary colors of light at the target diagnostic region captured at the current point in time, for each of the three primary colors.

10. The information terminal device according to claim 2, wherein
the detecting unit is configured to detect pigmented spots or tumorous areas which are present in the living body at the current point in time but were not present at a past point in time in the image that includes the target diagnostic region; and
the generating unit is configured to generate health management information for the living body by accounting for information on the pigmented spots or tumorous areas detected by the detecting unit.

11. The information terminal device according to claim 10, wherein
the image that includes the target diagnostic region captured by the imaging unit is a color image; and
the detecting unit is configured to detect appearance of the pigmented spots and tumorous areas in the living body based on a composite image which superimposes a first inverted image that inverts white and black portions of the entire image that includes the target diagnostic region at a past point in time converted from the color image to an achromatic image and a second inverted image that inverts the white and black portions of the entire image that includes the target diagnostic region at the current point in time.

12. The information terminal device according to claim 1, wherein
the imaging unit is configured such that a type of environmental light when capturing images that include the target diagnostic region is input; and
the detecting unit is configured to detect the amount of color change of the target diagnostic region in the second diagnostic data relative to the first diagnostic data by comparing the image that includes the target diagnostic region captured at a past point in time and the image that includes the target diagnostic region captured at the current point in time after performing color correction on the image that includes the target diagnostic region captured at a past point in time and/or the image that includes the target diagnostic region captured at the current point in time based on the type of environmental light that is input at each point in time.
